(19) United States
(12) Patent Application Publication
(10) Pub. No.: US 2008/0193424 A1
(43) Pub. Date: Aug. 14, 2008

(54) TREATMENT OF TISSUE DEFECTS WITH A THERAPEUTIC COMPOSITION

(75) Inventors: James M. McKale, Syracuse, IN (US); Joel C. Higgins, Claypool, IN (US); Matthew Swift, Fort Wayne, IN (US)

Correspondence Address: HARNESS, DICKEY & PIERCE, P.L.C., P.O. BOX 828, BLOOMFIELD HILLS, MI, 48303, US

(73) Assignee: Biomet Biologics, Inc., Warsaw, IN (US)

(21) Appl. No.: 12/028,361

(22) Filed: Feb. 8, 2008

Related U.S. Application Data
(60) Provisional application No. 60/900,758, filed on Feb. 9, 2007.

(51) Int. Cl. A61K 35/12

(52) U.S. Cl. 424/93.7

(57) ABSTRACT

Methods for treating a tissue defect include applying therapeutic compositions to the tissue defect. Treatment of a tissue defect, including wound healing can be enhanced by a process including obtaining blood compatible with the subject, fractionating the blood from the subject to produce a blood component, obtaining stromal cells from the subject, combining the stromal cells and the blood component to form a therapeutic composition, and delivering the therapeutic composition to the tissue defect. Therapeutic compositions for promoting wound healing include isolated stromal cells and a blood component.

### CROSS-REFERENCE TO RELATED APPLICATIONS

This application claims the benefit of U.S. Provisional Application No. 60/900,758 filed Feb. 9, 2007, the disclosure of which is incorporated herein by reference.

### INTRODUCTION

The present technology relates to methods and compositions for treating tissue defects to promote or enhance tissue repair.

There are a number of complex physiological steps and processes that are involved in tissue repair following a wound incident due to trauma, disease, surgery, or other disruption to tissue. Influencing these processes has been a major focus of medical research, and more effective compositions and methods to promote and enhance tissue repair in terms of ease of use, cost, healing rate and efficacy are desirable.

### SUMMARY

The present technology provides methods for treating a tissue defect comprising obtaining blood compatible with the subject, fractionating the blood to produce a blood component, obtaining stromal cells, combining the stromal cells and the blood component to form a therapeutic composition, and administering the therapeutic composition to the tissue defect. Some methods include obtaining stromal cells by performing lipoaspiration on the subject to obtain adipose tissue, enzymatically digesting the adipose tissue, and separating the adipose stromal cells from adipocytes. Some embodiments may also include obtaining stromal cells from bone marrow aspirate.

Further areas of applicability of the present technology will become apparent from the detailed description provided herein. It should be understood that the detailed description and specific examples, while indicating various embodiments of the technology described below, are intended for purposes of illustration only and are not intended to limit the scope of the teachings.

### BRIEF DESCRIPTION OF THE DRAWINGS

The present technology will become more fully understood from the detailed description and the accompanying drawings, wherein:

FIG. 1 illustrates a representative site of a tissue defect on a subject in need of treatment according to one embodiment of the present technology;

FIG. 2 is a diagrammatic illustration of a representative method for treating a tissue defect according to one embodiment of the present technology;

FIG. 3 is a cross-sectional view of a representative device used for isolating a blood component according to one embodiment of the present technology;

FIGS. 4A and 4B are cross-sectional views of a representative device used for concentrating platelet-poor plasma or other blood components;

FIG. 5 is a partial cross-sectional view of a representative device for isolating stromal cells from adipose tissue, bone marrow aspirate, and/or other tissue suspension;

FIG. 6 is a diagrammatic illustration of a representative method for washing stromal cells; and

FIG. 7 illustrates a representative manner of administering a therapeutic composition to the subject according to one embodiment of the present technology.

### DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom.

Referring to FIG. 1, a tissue defect represented by a soft tissue wound 10 on a foot 12 of a human subject is shown. The soft tissue wound 10 may be an ulcer (e.g., a venous ulcer, pressure ulcer, or diabetic ulcer), and it may be located on a body extremity or elsewhere on the body of the subject, such as on the torso or head. It should be understood, however, that the tissue defect may be any condition in which tissue (including hard and soft tissues) is inadequate for physiological or cosmetic purposes. In this regard, the tissue defect may include congenital tissue defects, tissue defects that result from or are symptomatic of disease, disorder, or trauma, and those tissue defects that are consequent to surgical or other medical procedures. For example, tissue defects may be defects resulting from osteoporosis, spinal fixation procedures, hip and other joint replacement procedures, chronic wounds, wounds following chemotherapy or radiotherapy, diabetic ulcers or other ulcers (as discussed above), myocardial infarction, peripheral vascular disease, fractures, sclerosis of tissues and muscles, Alzheimer's disease, Parkinson's disease, and spinal cord or other nerve injury.

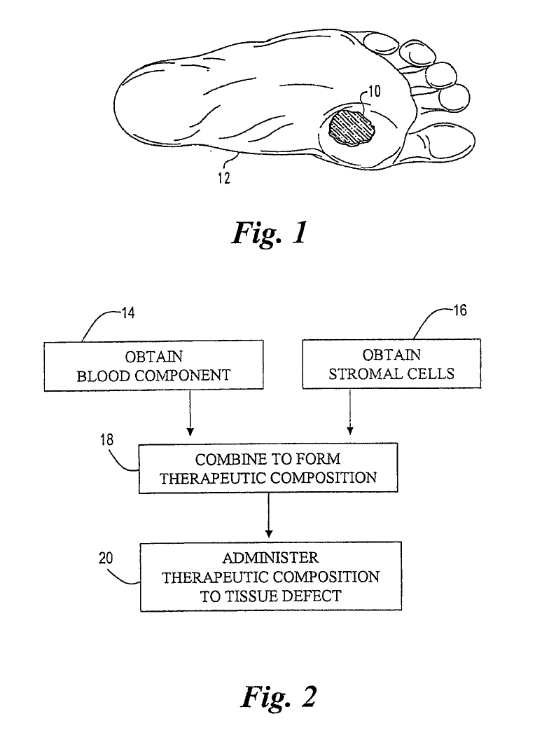

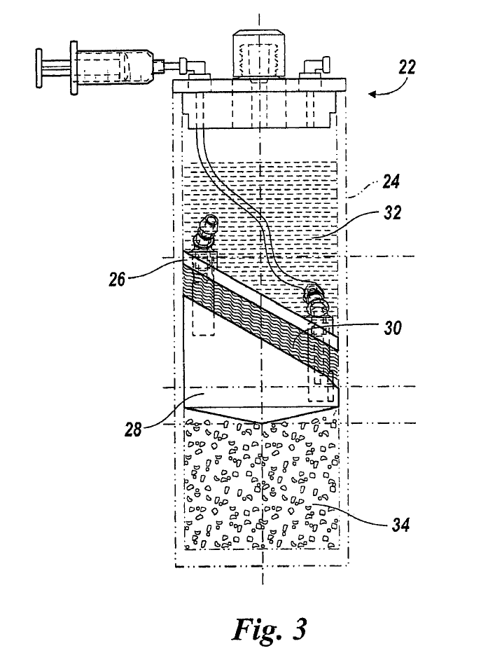

One embodiment for treating a tissue defect is shown diagrammatically in FIG. 2. In summary, a blood component is obtained at step 14. Stromal cells, such as adipose and bone marrow derived stromal cells, are obtained at step 16. The blood component obtained at step 14 and the stromal cells obtained at step 16 are combined in step 18 to form a therapeutic composition. Optional components, such as one or more additives described below, may also be included in the therapeutic composition at step 18. The therapeutic composition formed at step 18 is then administered to the site of the tissue defect at step 20. In various embodiments, the blood component and the stromal cells act in concert to more effectively treat a tissue defect than when used individually. In this regard, administration of the therapeutic composition results in enhanced healing of the tissue defect and/or more complete healing of the tissue defect compared to treatment using either the blood component or stromal cells alone. Each of these steps will be more fully discussed below.

In particular, a blood component is initially obtained at step 14. The blood component may be isolated from the subject exhibiting the tissue defect, or the blood component may be isolated from donor blood identified as being compatible with the subject. The blood component may comprise fractionated plasma in the form of platelet-rich plasma, platelet-poor plasma, or concentrated platelet-poor plasma. In this regard, the blood component comprising platelet-rich plasma may have an increased concentration of platelets relative to whole blood, and in some embodiments, the platelet concentration can be from about 3-fold to about 10-fold greater than the platelet concentration in whole blood. In addition, the blood component comprising platelet-poor plasma may have a decreased concentration of platelets relative to whole blood, and in some embodiments, the platelet concentration can be from about 0 to about 100,000 platelets/microliter. The platelet-poor plasma can also be concentrated to make concentrated platelet-poor plasma. In some embodiments, platelet-poor plasma and concentrated platelet-poor plasma can contain substantially no platelets.

In addition, step 14 may also include obtaining a blood component comprising combinations of fractionated plasma. For example, the blood component may have varying proportions of platelet-rich plasma and platelet-poor plasma, resulting in ranges of platelet concentrations that are continuous from platelet-rich plasma to platelet-poor plasma. The blood component may also comprise platelet-rich plasma or isolated platelets, either of which may be diluted and/or resuspended with platelet-poor plasma or concentrated platelet-poor plasma. Embodiments also include using whole blood as the blood component. In some cases, the blood component may further include polyethylene glycol and/or albumin.

One example of a device that may be used for forming the blood component at step 14 is shown in FIG. 3. In this regard, the device 22 includes a container 24, such as a tube, that is placed in a centrifuge after being filled with blood. The container 24 includes a buoy system having an isolator 26 and a buoy 28. The buoy 28 has a selected density which is tuned to reach a selected equilibrium position upon centrifugation; this position lies between a more dense blood fraction and a less dense blood fraction. During centrifugation, the buoy 28 separates the blood within the container 24 into at least two fractions, without substantially commingling the fractions, by sedimenting to a position between the two fractions. In this regard, the isolator 26 and the buoy 28 define a layer comprising platelet-rich plasma 30, while less dense platelet-poor plasma 32 generally fractionates above the isolator 26, and more dense red blood cells 34 generally fractionate below the buoy 28. Following centrifugation, a syringe or tube may then be interconnected with a portion of the buoy system to extract one or more selected fractions for use as the blood component. One such device that is commercially available is the GPS® Platelet Concentrate System, from Biomet Biologics, Inc. (Warsaw, Ind.).

Other methods may be used to obtain the blood component in step 14. For example, whole blood can be centrifuged without using a buoy system, whole blood may be centrifuged in multiple stages, continuous-flow centrifugation can be used, and filtration can also be used. In addition, a blood component including platelet-rich plasma can be produced by separating plasma from red blood cells using a slow speed centrifugation step to prevent pelleting of the platelets. In other embodiments, the buffy coat fraction formed from centrifuged blood can be separated from remaining plasma and resuspended to form platelet-rich plasma. In additional embodiments, platelet-poor plasma can be produced by separating plasma from red blood cells using a high speed centrifugation step to pellet platelets with the red blood cells, and then the non-pelleted fraction can be used as platelet-poor plasma.

Devices that may be used to obtain the blood component at step 14 are described, for example, in U.S. Pat. No. 6,398,972; U.S. Pat. No. 6,649,072; U.S. Pat. No. 6,790,371; U.S. Pat. No. 7,011,852; U.S. Patent Application Publication No. 2004/0251217 (incorporated by reference herein); U.S. Patent Application Publication No. 2005/0109716 (incorporated by reference herein); U.S. Patent Application Publication No. 2005/0196874 (incorporated by reference herein); and U.S. Patent Application Publication No. 2006/0175242 (incorporated by reference herein).

In addition to the GPS® Platelet Concentrate System, a variety of other commercially available devices may be used to obtain the blood component at step 14, including the Magellan™ Autologous Platelet Separator System, commercially available from Medtronic, Inc. (Minneapolis, Minn.); SmartPReP™, commercially available from Harvest Technologies Corporation (Plymouth, Mass.); DePuy (Warsaw, Ind.); the AutoloGel™ Process, commercially available from Cytomedix (Rockville, Md.); the GenesisCS System, commercially available from EmCyte Corporation (Fort Myers, Fla.); and the PCCS System, commercially available from Biomet 3i, Inc. (Palm Beach Gardens, Fla.).

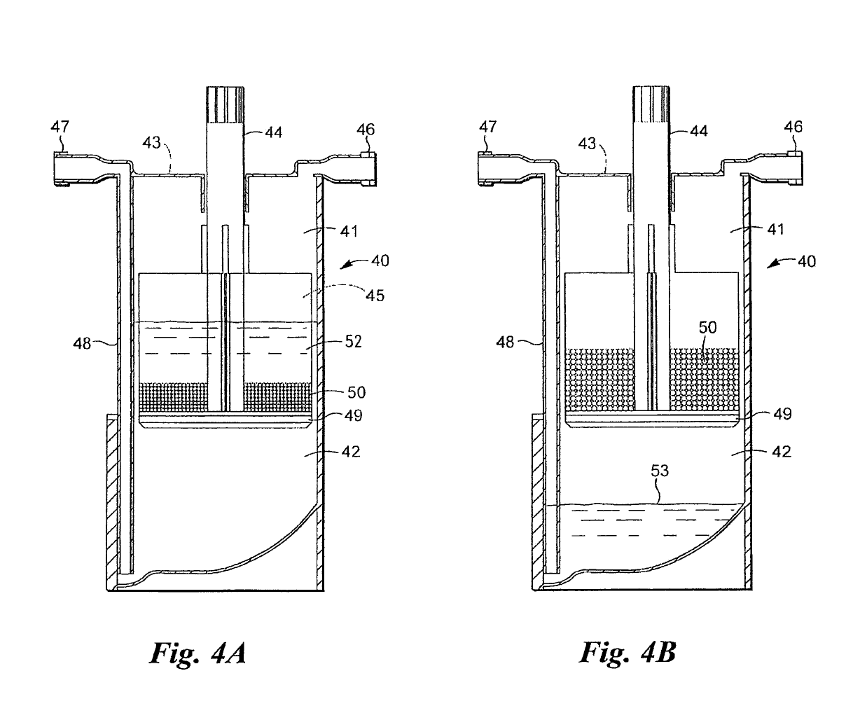

A concentrated blood component, such as concentrated platelet-poor plasma, may also be obtained at step 14. One example of a device that may be used for forming concentrated platelet-poor plasma at step 14 is shown in FIGS. 4A and 4B. In this regard, the device 40 has an upper chamber 41 and a lower chamber 42. The upper chamber 41 has an end wall 43 through which the agitator stem 44 of a gel bead agitator 45 extends. The device 40 also has a plasma inlet port 46 that extends through the end wall 43 and into the upper chamber 41. The device 40 also includes a plasma concentrate outlet port 47 that communicates with a plasma concentrate conduit 48. The floor of upper chamber 41 includes a filter 49, the upper surface of which supports desiccated concentrating polyacrylamide beads 50.

During use, blood plasma 52 (preferably cell free) is initially introduced into the upper chamber 41 through the plasma inlet port 46. The plasma 52 entering the upper chamber 41 flows to the bottom of the chamber where it contacts the polyacrylamide beads 50 as shown in FIG. 4A. As the polyacrylamide beads 50 remove water from plasma 52, the plasma proteins are concentrated. During this concentration stage, the plasma and its components can be concentrated to a concentration from about 1.5 to about 3 times or higher than its original concentration.

Referring to FIG. 4B, the device 40 is then placed in the cup receptors of a conventional laboratory centrifuge (not shown) and spun at a speed that will create a force that will remove plasma concentrate 53 from the polyacrylamide beads 50, and cause the plasma concentrate 53 to flow through the filter 49. The filter 49 can be constructed to allow flow of liquid there-through at forces above 10 g. After centrifugation is completed, the device 40 is removed from the centrifuge. The plasma concentrate 53 is then drawn from lower chamber 42 through conduit 48 to the plasma concentrate outlet 47.

Exemplary plasma concentration devices are disclosed in U.S. Patent Application Publication 2006/0175268, Dorian et al., published Aug. 10, 2006; and U.S. Patent Application Publication 2006/0243676, Swift et al., published Nov. 2, 2006; both of which are incorporated by reference herein. Such a device is commercially available as Plasmax™ Plus Plasma Concentrator, from Biomet Biologics, Inc. (Warsaw, Ind.).

Referring again to FIG. 2, stromal cells, which can be adipose derived stromal cells, bone marrow derived stromal cells, or combinations thereof, are obtained as shown in step 16. The stromal cells may be mammalian cells, and may be human embryonic or adult cells derived from embryonic or adult tissues, respectively. The stromal cells can be autologous, allogenic, or combinations thereof. Preferably, the stromal cells are autologous. The stromal cells that are obtained in step 16 may include cells capable of differentiating into one or more of the following cell types: chondrocytes, endothelial cells, osteoblasts, myocytes, neural cells, glial cells, adipocytes, pericytes, cardiomyocytes, epithelial cells, fibroblasts, and combinations thereof.

Step 16 may include obtaining the stromal cells by disaggregating an appropriate organ or tissue which is to serve as the source of the stromal cells, such as adipose tissue or bone marrow. For example, the tissue or organ can be disaggregated mechanically and/or enzymatically. In one method, adipose tissue is extracted by standard liposuction and lipoaspiration methods known in the art to harvest and disaggregate adipose tissue. Adipose tissue may also be treated with digestive enzymes and with chelating agents that weaken the connections between neighboring cells, making it possible to disperse the tissue into a suspension of individual cells without appreciable cell breakage. In another method, bone marrow derived stromal cells may be isolated from bone marrow tissue, including bone marrow aspirate harvested by methods known in the art. Methods may also include compositions of stromal cells comprising both adipose stromal cells and bone marrow derived stromal cells. Stromal cells from adipose and bone marrow tissues may be obtained from the same organism or from different organisms.

Following disaggregation, the stromal cells may be isolated from the suspension of cells and disaggregated tissue, such as adipose tissue or bone marrow aspirate. A device similar to (or the same device) that is shown in FIG. 3 can be used, such as the GPS® Platelet Concentrate System. In this regard, the suspension of cells is placed in the container 24 having an isolator 26 and a buoy 28. Whole blood may also be added to the suspension of cells and the container 24 centrifuged. The buoy 28 separates the suspension of cells (and the blood if included) during centrifugation into at least two fractions without substantially commingling the fractions. The stromal cells sediment between the isolator 26 and the buoy 28. In the case where blood is combined with the cell suspension, the stromal cells co-sediment with the platelet-rich plasma 30. Thus, the isolator 26 generally separates the platelet-rich plasma 30 and the stromal cells from platelet-poor plasma 32, and the buoy 28 generally separates platelet-rich plasma 30 and the stromal cells from the red blood cells 34. Following centrifugation, a syringe or tube may then be interconnected with a portion of the buoy system to extract the stromal cells and platelet-rich plasma. This mixture may be used as a therapeutic composition, as described below in reference to step 18, or may be subjected to additional treatments, such as washing of the cells and/or combination with another blood component, as is further described below. Moreover, the mixing of stromal cells and whole blood followed by centrifugation may be effective to wash the stromal cells, as discussed below.

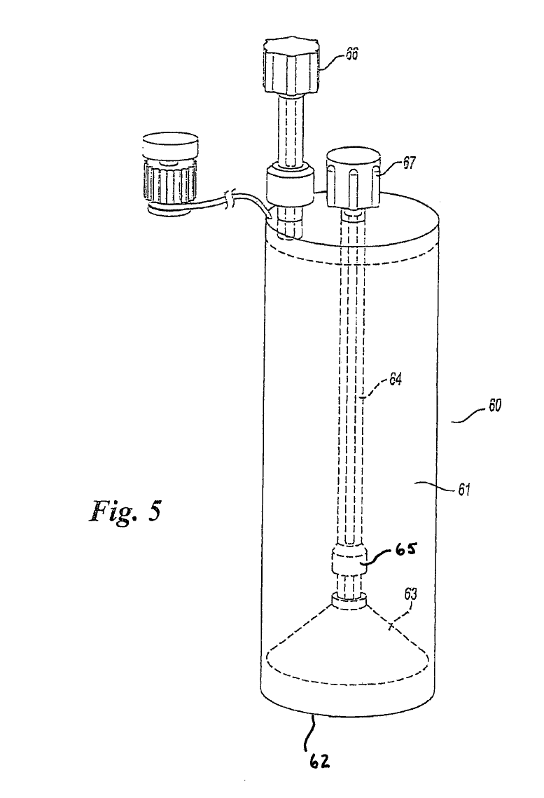

Another example of a device that may be used for obtaining stromal cells at step 16 is shown in FIG. 5. In this regard, the device 60 can isolate stromal cells from a cell suspension formed from disaggregated tissue. Other fluids, including a blood component as obtained in step 14 or whole blood, may be combined with the suspension of cells. The cell suspension, along with any additional fluid(s), is loaded into a separation container 61 having a piston 63 and a withdrawal tube 64. The withdrawal tube 64 may run the length of the separation container 61, from top to bottom 62 (i.e., the direction in which force is applied when spinning the tube in a centrifuge), while piston 63 can be slidably engaged about the withdrawal tube 64. There may be a stop 65 on the tube to limit the upward movement of the piston 63. (Piston 63 is shown slightly elevated along withdrawal tube 64, so as to show the bottom 62 of the separation container 61.) In use, a cell suspension is introduced to separation container 61 through an injection port 66. During centrifugation, piston 63 moves relative to the density of the cell suspension by sliding upward along withdrawal tube 64. The stromal cells sediment to the bottom 62 of the separation container 61 forming a cell pellet. The cells are then extracted from separation container 61 through withdrawal tube 64, after removing the cap 67 from the top of the withdrawal tube 64. Removal of cells may be aided by injecting liquid into the separation container 61 through the withdrawal tube 64, so as to dislodge the cells from the bottom 62 of the separation container 61. An example of a device useful at step 16 is the LipoStim™ biomaterial separation unit (Biomet Biologics, Warsaw, Ind.) or a similar centrifugation-based cell isolation device as described in U.S. application Ser. No. 11/210,005 filed Aug. 23, 2005.

Step 16 may also include using a centrifuge to isolate stromal cells from the cell suspension. Centrifugation may include spinning the suspension of cells so that the cells form a pellet including the stromal cells, the supernatant may then be siphoned off, and the cells may be resuspended in one or more fluids, such as a blood component as described above in reference to step 14, or a washing fluid as further described below.

Step 16 may also include obtaining stromal cells from established cell lines, or from lipoaspiration or liposuction of adipose tissue from a suitable mammalian source. Such methods include those disclosed in U.S. Pat. No. 6,355,239 and U.S. Pat. No. 6,541,024. Combinations of adipose stromal cells may be used from two or more sources; in addition, these combinations may also include bone marrow derived stromal cells.

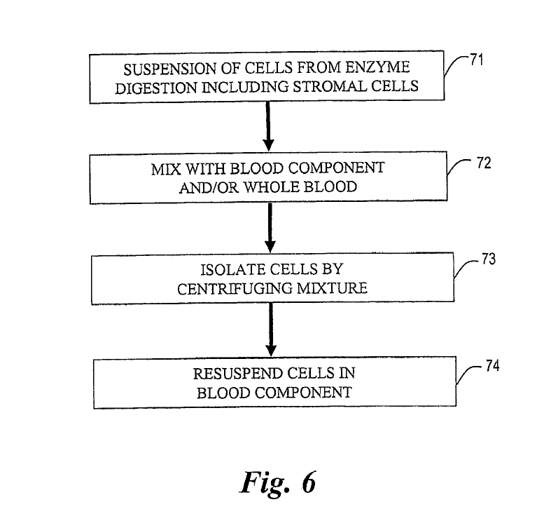

Step 16 may also include washing the stromal cells to remove, dilute, and/or inactivate digestive enzymes used in the isolation of the stromal cells. A general procedure for washing the stromal cells is exemplified in FIG. 6. In step 71, a suspension or other mixture of stromal cells is obtained as described above. In step 72, the suspension is then mixed with a fluid, such as saline; whole blood; a blood component including platelet-poor plasma, concentrated platelet-poor plasma, or platelet-rich plasma; cryoprecipitated plasma; bone marrow aspirate; or combinations thereof. These fluids may also be derived from an autologous or allogenic source using methods including those described above for creating a blood component in reference to step 14. For example, platelet-rich plasma and platelet-poor plasma may be obtained using the GPS® Platelet Concentrate System and concentrated platelet-poor plasma may be obtained using the Plasmax™ Plus Plasma Concentrator, as previously described above. Such fluids can be used in place of washing with phosphate buffered saline. Such fluids can also be used in place of washing with bovine serum and/or bovine albumin, which are commonly used in the art to remove and/or inactivate enzymes, thereby avoiding use of materials of bovine origin.

As discussed above, the mixture of stromal cells and blood component (such as platelet-poor plasma), produced in step 72 can be used as a therapeutic composition in step 18 without performing steps 73 and 74. In other embodiments, the mixture of the stromal cells and blood component are extracted in step 73, following centrifugation, and can be used as a therapeutic composition in step 18 without performing step 74. In another embodiment, the blood component is mixed with the stromal cells in order to wash the cells and dilute and/or inactivate enzymes that were used in processing the stromal cells. The stromal cells are isolated by centrifugation in step 73, and the majority of the supernatant removed. After isolation of the stromal cells in step 73, the cells may be resuspended in step 74, in a blood component or other suitable fluid via agitation.

Washing of stromal cells may also include the use of a buoy system, such as the GPS® Platelet Concentrate System. In this regard, washing of stromal cells comprises mixing stromal cells and whole blood in step 72, by adding both materials to a container having a buoy. The container is then centrifuged so that the buoy defines an interface between platelet-rich plasma and platelet-poor plasma, with the stromal cells co-sedimenting with the platelet-rich plasma. The layer containing the washed stromal cells and platelet-rich plasma is then removed from the container and may be used as a therapeutic composition in step 18 as discussed below.

Another method includes washing stromal cells with platelet-poor plasma. In this method, whole blood may be fractionated to isolate platelet-poor plasma and platelet-rich plasma; the GPS® Platelet Concentrate Separation Kit may be used to isolate these blood components, as described above. Stromal cells are added to the isolated platelet-poor plasma to wash the cells and this combination is centrifuged again in the same GPS® device. The sedimenting fraction containing the washed stromal cells is effectively combined with the platelet-rich plasma layer from the initial whole blood fractionation to thereby form a therapeutic composition.

Washing of stromal cells may also include mixing of stromal cells and a fluid, such as saline or platelet-poor plasma, in a device shown in FIG. 5. For example, platelet-poor plasma may be obtained by using the GPS® Platelet Concentrate Separation Kit discussed above. The stromal cells and platelet-poor plasma may then be combined in separation container 61 and centrifuged so that the stromal cells sediment into a cellular pellet. The platelet-poor plasma supernatant is then removed, and the cellular pellet is resuspended with a blood component in step 74, to form a therapeutic composition as discussed below.

Step 16 may also include washing the resuspended stromal cells one or more additional times using any of the aforementioned methods, including centrifuging to form a cellular pellet, removing the supernatant, and resuspending the stromal cells in one or more fluids. In some embodiments, the fluid used in each washing step may be the same fluid, or a different fluid can be used in one or more successive washing steps. For example, the cellular pellet formed while washing stromal cells using the device of FIG. 5 may be mixed with whole blood and further washed using a GPS® Platelet Concentrate System or similar device, as discussed above.

Referring again to FIG. 2, the blood component obtained in step 14 is combined with the stromal cells obtained in step 16 to form a therapeutic composition as indicated by step 18. In some embodiments, the combination of stromal cells and blood component in step 18 is formed while obtaining the blood component of step 14. For example, as discussed above, stromal cells may be mixed with whole blood. The stromal cells may be obtained from enzymatic digestion, as discussed above, and may also be washed prior to mixing, such as by using platelet-poor plasma. The mixture of stromal cells and whole blood is then added to a separation device, such as the GPS® Platelet Concentrate Separation Kit, and centrifuged in order to isolate a layer containing the stromal cells and platelet-rich plasma. As also discussed above, such mixing and centrifugation of the whole blood and stromal cells may act to wash the cells and further dilute and/or inactivate the digestive enzyme if present.

In one embodiment, step 18 comprises mixing stromal cells with concentrated platelet-poor plasma, thereby forming a therapeutic composition. This may be performed using the device of FIG. 4, such as the Plasmax™ Plus Plasma Concentrator device. Referring to FIG. 4, in one embodiment stromal cells are injected via the port 47 to the lower chamber 42 of the device 40 via a syringe, and platelet-poor plasma is injected to upper chamber 41 via the port 46. Following stirring with the agitator 45, the device 40 is centrifuged, forming a mixture 53 of concentrated platelet-poor plasma and stromal cells in the lower chamber 42. In another embodiment, concentrated platelet-poor plasma is obtained by injecting platelet-poor plasma to upper chamber 41, centrifuging the device 40 to form concentrated platelet-poor plasma 53 in the lower chamber 42, and then injecting stromal cells to the bottom chamber 42 via the port 47. In another embodiment, concentrated platelet-poor plasma is obtained by injecting platelet-poor plasma to the upper chamber 41, centrifuging the device 40 to form concentrated platelet-poor plasma 53 in the lower chamber 42, and then a syringe containing stromal cells is used to extract concentrated platelet-poor plasma from the device 40 via the port 47 thereby combining the cells and platelet-poor plasma within the syringe.

Step 18 may further include the addition of a platelet activator, scaffold, bioactive material, cytokine, conditioned cell culture medium, polyethylene glycol, buffer, or combinations thereof to the therapeutic composition. In this regard, the platelet activator may serve to activate one or more growth factors within platelets contained in a blood component. Activation of the platelets by the platelet activators can be performed just prior to administration of the therapeutic composition, concomitant with administration of the therapeutic composition, or following administration of the therapeutic composition to the site. Platelet activators among those useful herein include thrombin (such as autologous thrombin), calcium chloride, coagulation factors, and mixtures thereof. Coagulation factors include, but are not limited to, one or more of the following: V, VII, VIIa, IX, IXaβ, X, Xa, XI, XIa, XII, α-XIIa, β-XIIa, and XIII.

As discussed above, step 18 may also include the addition of a scaffold that contains or supports stromal cells, preferably enabling their growth and/or retention at the site of implantation. When a scaffold is included in step 18, the scaffold may be implanted at the tissue defect site to which the therapeutic composition is subsequently administered. Alternatively, the therapeutic composition is combined with the scaffold prior to implantation.

Scaffolds may be formed from porous or semi-porous, natural, synthetic or semisynthetic materials. Scaffold materials include those selected from the group consisting of bone (including cortical and cancellous bone), demineralized bone, ceramics, polymers, fibrin sealant, and combinations thereof. Suitable polymers may include collagen, gelatin, polyglycolic acid, polylactic acid, polypropylenefumarate, polyethylene glycol, and copolymers or combinations thereof. Ceramics include any of a variety of ceramic materials known in the art for use for implanting in bone, including calcium phosphate (including tricalcium phosphate, tetracalcium phosphate, hydroxyapatite, and mixtures thereof). Ceramics useful herein include those described in U.S. Pat. No. 6,323,146 and U.S. Pat. No. 6,585,992. A commercially available ceramic is ProOsteon™ from Interpore Cross International, Inc. (Irvine, Calif.).

Step 18 may also include the addition of one or more bioactive materials that provide a therapeutic, nutritional or cosmetic benefit to the subject in which the therapeutic compositions of the present technology are applied. Such benefits may include repairing unhealthy or damaged tissue, minimizing infection at the implant site, increasing integration of healthy tissue into the medical implant, and preventing disease or defects in healthy or damaged tissue. The bioactive materials can be applied to the site just prior to the administration of the therapeutic composition, concomitant with administration the therapeutic composition, or following administration of the therapeutic composition to the subject.

Bioactive materials that may be included in step 18 include organic molecules, proteins, peptides, peptidomimetics, nucleic acids, nucleoproteins, antisense molecules, polysaccharides, glycoproteins, lipoproteins, carbohydrates and polysaccharides, and synthetic and biologically engineered analogs thereof, living cells (other than stromal cells) such as chondrocytes, bone marrow cells, viruses and virus particles, natural extracts, and combinations thereof. Specific non-limiting examples of bioactive materials include hormones, antibiotics and other antiinfective agents, hematopoietics, thrombopoietics, agents, antidementia agents, antiviral agents, antitumoral agents (chemotherapeutic agents), antipyretics, analgesics, antiinflammatory agents, antiulcer agents, antiallergic agents, antidepressants, psychotropic agents, anti-parkinsonian agents, cardiotonics, antiarrythmic agents, vasodilators, antihypertensive agents, diuretics, anti-cholinergic, antidiabetic agents, anticoagulants, cholesterol lowering agents, gastrointestinal agents, muscle relaxants, therapeutic agents for osteoporosis, enzymes, vaccines, immunological agents and adjuvants, cytokines, growth factors, cellular attractants and attachment agents, gene regulators, vitamins, minerals and other nutritionals, and combinations thereof.

Step 18 may also include the addition of one or more cytokines, including isolated, synthetic or recombinant molecules. Cytokines useful herein include growth factors such as transforming growth factor (TGF-beta), bone morphogenic proteins (BMP, BMP-2, BMP-4, BMP-6, and BMP-7), neurotrophins (NGF, BDNF, and NT3), fibroblast growth factor (FGF), granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), growth differentiation factor-9 (GDF9), basic fibroblast growth factor (bFGF or FGF2), vular endothelial growth factor (VEGF), epidermal growth factor (EGF), insulin-like growth factors (IGF-I, IFG-II), and combinations thereof. Cytokines can be applied to the site just prior to the administration of the therapeutic composition, concomitant with administration the therapeutic composition, or following administration of the therapeutic composition to the subject.

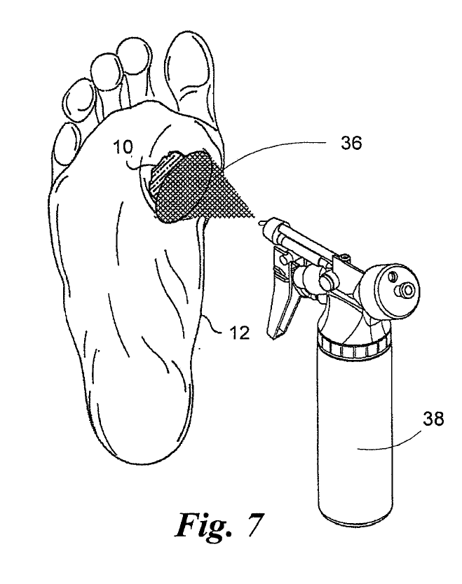

Referring again to FIG. 2, the therapeutic composition formed at step 18 is administered to the tissue defect at step 20. In this regard, the therapeutic composition 36 can be sprayed onto the soft tissue wound 10 using a spray applicator 38 as shown in FIG. 7. It should be understood, however, that administering the therapeutic composition may comprise any biomedically acceptable process or procedure by which the therapeutic composition is implanted, injected, sprayed, applied, or otherwise administered in, on, or in proximity to the site of the tissue defect so as to have a beneficial effect to the tissue. For example, methods of treating a tissue defect may include applying the therapeutic composition to a surgical site to facilitate or enhance the rate of healing and/or provide for more complete healing. In some embodiments, administration in step 18 comprises multiple applications of a therapeutic composition in a regular or irregular pattern in and surrounding the site of the tissue defect.

Treatment of tissue defects using the present technology may be evaluated by a variety of techniques, including measuring surface wound healing, where applicable, and by histological and immunohistological observation. For external wounds, wound contraction may be followed using digital calipers, where treatment effectiveness can be expressed as the percentage of reduction of the original wound area. Effective wound treatment may also be evaluated by scoring wound color, smoothness, and wound suppleness or stiffness.

The following non-limiting examples illustrate the compositions, methods, and processes of the present technology. The examples are provided for illustrative purposes of how to make and use the compositions and methods of this technology and, unless explicitly stated otherwise, are not intended to be a representation that given embodiments of this technology have, or have not, been made or tested.

### EXAMPLE 1

Compositions containing adipose stromal cells and blood components (platelet-rich plasma or platelet-poor plasma) are made as follows. Adipose tissue is minced into small pieces (˜1 cm³) and digested in 2 mg/ml type I collagenase (Worthington Biochemical Corp., Lakewood, N.J.) under intermittent mechanical agitation in a water bath at 37° C. for 180 minutes. Digestion is neutralized by the addition of medium or blood-derived solution. This cell suspension is centrifuged (300 g for 7 minutes at 25° C.) followed by removal of the supernatant and resuspension of the cell pellet in new solution.

Platelets are separated from 55 ml of autologous peripheral blood obtained from the femoral artery mixed with an anticoagulant citrate dextrose solution A (Citra Anticoagulants, Inc., Braintree, Mass.) in a GPS® Platelet Concentrate System, from Biomet Biologics, Inc. (Warsaw, Ind.). The blood is separated via a single, 15-minute centrifuge spin, which collects approximately 6 ml of platelet-rich plasma and 30 ml of platelet-poor plasma per 55 ml of separated whole blood. The platelet-rich plasma, platelet-poor plasma, and autologous adipose stromal cells are used to create two compositions (adipose stromal cells with platelet-poor plasma, and adipose stromal cells with platelet-rich plasma). Each composition is placed into a FibriJet® surgical sealant applicator (Micromedics, Inc., St. Paul, Minn.). A 20 ga FibriJet® dual cannula applicator tip is placed on each applicator. A solution of 5000 IU topical thrombin (Jones Pharma Inc, Bristol, Va.) and 5 ml of 10% calcium chloride is placed into each applicator using a 10:1 ratio of adipose stromal cell-containing treatment composition to the thrombin/calcium chloride solution.

Each composition (adipose stromal cells with platelet-poor plasma, and adipose stromal cells with platelet-rich plasma) is administered by spraying on the foot ulcer. Within about three weeks after administration, the ulcer reveals significant vascularization and wound healing maturation.

### EXAMPLE 2

A therapeutic composition for treating tendinosis associated with plantar fasciitis in a human subject is made containing isolated autologous adipose stromal cells and autologous platelet-rich plasma. The adipose stromal cells are obtained by harvesting adipose tissue from the subject, and isolated using a device of FIG. 5 to form a pellet of adipose stromal cells. The pellet is then suspended with whole blood obtained from the subject, and added to a GPS® Platelet Concentrate System, from Biomet Biologics, Inc. (Warsaw, Ind.). Following centrifugation, the platelet-rich plasma layer, which contains the adipose stromal cells, is extracted from the system. This mixture is then added to a solution containing 5,000 units topical thrombin in 5 ml of 10% calcium chloride, at a 10:1 volume ratio. The thrombin and CaCl₂ solution activates the platelets in the therapeutic composition. Approximately 8 injections of the resulting therapeutic composition are made into the tendon, with each injection containing about 0.75 ml of composition. Within about eight weeks after administration, the pain associated with the fasciitis has decreased significantly.

### EXAMPLE 3

In a method of repairing a defect in cartilage at the knee of a human subject, a therapeutic composition is made containing adipose stromal cells and autologous platelet-rich plasma. Isolation of human adipose stromal cells is performed by obtaining human subcutaneous adipose tissue from lipoaspiration/liposuction procedures and digesting the tissue in collagenase type I solution (Worthington Biochemical Corp., Lakewood, N.J.) under gentle agitation for 1 hour at 37° C. The dissociated cells are filtered with 500-μm and 250-μm Nitex filters, and centrifuged at 200 g for 5 minutes to separate the stromal cell fraction (pellet) from the adipocytes. The fraction is centrifuged at 300 g for 5 minutes. The supernatant is discarded, and the cell pellet is resuspended in a blood-derived solution.

Blood is also obtained from the subject, and processed using a GPS® Platelet Concentrate System from Biomet Biologics, Inc. (Warsaw, Ind.). The platelet-rich plasma is obtained, and mixed with the adipose stromal cells. The composition is injected into the cartilage at multiple sites. After four weeks, the cartilage shows significant radiographic improvement.

### EXAMPLE 4

A process for treating a wound associated with chemotherapy and/or radiation therapy of a cancerous tumor in a human subject uses a composition containing adipose stromal cells and concentrated platelet-poor plasma. Adipose stromal cells are harvested from the subject by lipoaspiration of adipose tissue, the adipose tissue is enzymatically digested, and the adipose stromal cells are separated from the adipocytes. Blood is drawn from the subject and fractionated to produce platelet-poor plasma that is further concentrated using a Plasmax™ Plus Plasma Concentrator, from Biomet Biologics, Inc. (Warsaw, Ind.). The adipose stromal cells are injected into the concentrated platelet-poor plasma while in the concentrator. The mixture of adipose stromal cells and concentrated platelet-poor plasma is then extracted from the concentrator. The therapeutic composition is applied to the site of the wound. The wound is observed to be substantially healed in less than three weeks. In the above example, the composition may be applied to a surgical mesh or other scaffold at the site of the wound, with substantially similar results.

### EXAMPLE 5

A therapeutic composition is made containing adipose stromal cells and platelet-rich plasma, for treatment of peripheral vascular disease in a human subject. The adipose stromal cells are obtained by processing of adipose tissue from the subject. The adipose tissue is mechanically processed, and placed in the top chamber of a device of FIG. 5. Enzyme is then added to the top chamber to digest the tissue. Following an incubation period, the device is centrifuged to form a cell pellet in the bottom chamber of the device. A majority of the supernatant is discarded. The cell pellet, containing adipose stromal cells, is then suspended in the remaining supernatant, and extracted from the device. The adipose stromal cells are placed into the top chamber of the second device of FIG. 5.

Separately, whole blood is obtained from the subject and processed using a GPS® Platelet Concentrate System from Biomet Biologics, Inc. (Warsaw, Ind.). Platelet-rich plasma and platelet-poor plasma are obtained. The platelet-poor plasma is then injected into the top chamber of the second device and combined with the adipose stromal cells to wash the cells and inactivate enzymes used in the processing of the adipose tissue. The device is centrifuged to create a pellet of cells. The cells are then extracted from the device, and mixed with the previously obtained platelet-rich plasma. The resulting therapeutic composition is administered to the site of the vascular disease in a series of forty 0.75 ml injections, using a 26-gauge needle at a depth of about 1.5 cm, in a 3×3 cm grid-like pattern. Significant improvement in vascularization results in about four weeks.

### EXAMPLE 6

A therapeutic composition is made containing bone marrow stromal cells and platelet-rich plasma, for the treatment of cardiac muscle damage following myocardial infarct in a human subject. Bone marrow aspirate is obtained from the patient's posterior iliac crest via a bone marrow aspiration needle. Bone marrow stromal cells are obtained by processing the bone marrow aspirate in a device similar to (or the same as) the device of FIG. 3. The bone marrow aspirate is placed in the container having an isolator and a buoy, and the container is centrifuged. The buoy separates the bone marrow aspirate during centrifugation into at least two fractions without substantially commingling the fractions. The stromal cells sediment between the isolator and the buoy. Following centrifugation, the isolated bone marrow stromal cells are extracted. Separately, whole blood is obtained from the subject and processed using a GPS® Platelet Concentrate System from Biomet Biologics, Inc. (Warsaw, Ind.). Platelet-rich plasma and platelet-poor plasma is obtained. The platelet-rich plasma is combined with the bone marrow stromal cells to form a therapeutic composition. The composition is then injected at ten sites, in a grid-like pattern in the area of the infarct. Each injection contains about 1 ml of composition. The subject is observed to have significantly improved cardiac function within five weeks of treatment. In the above Example, the composition is administered, with substantially similar results, using a device and method disclosed in U.S. Pat. No. 6,432,119, Saadat, issued Aug. 13, 2002, incorporated herein by reference.

Benefits of the present technology include promoting and/or enhancing healing of a tissue defect relative to an untreated tissue defect. Enhanced healing includes increasing the rate of healing and angiogenesis and/or providing more complete healing. In some embodiments, administration of the therapeutic composition has an elongated effect when compared to the effect of either the blood component or stromal cells administered separately. Embodiments of therapeutic compositions may also operate to modulate the activity of the stromal cells in whole or in part. In this regard, the therapeutic compositions modify one or more activities of the stromal cells by, for example, enhancing or increasing the rate, duration or magnitude of such activities as proliferation, production of molecules normally produced by the stromal cells (such as growth factors), and differentiation of the stromal cells into differentiated cell types.

In some embodiments, without limiting the mechanism, function or utility of the present technology, combinations of a blood component (for example, platelet-rich plasma) and isolated stromal cells, afford synergistic results. In various embodiments, the blood component and isolated stromal cells are administered at “synergistic” levels. Accordingly, the therapeutic effect of administering of the combination of the components in such embodiments is greater than the additive effect of administering the blood component and stromal cells individually. Such effects include one or more of increasing vessel density, enhanced local concentrations of growth factors (for example TGF-B1, PDGF-BB, VEGF, and EFG), improved wound healing, and reduced pain.

The examples and other embodiments described herein are exemplary and not intended to be limiting in describing the full scope of compositions and methods of this technology. Equivalent changes, modifications and variations of specific embodiments, materials, compositions and methods may be made within the scope of the present technology, with substantially similar results.

What is claimed is:

1. A method for treating a tissue defect in a human subject comprising:
obtaining blood compatible with the subject;
fractionating said blood to produce a blood component, said blood component selected from the group consisting of platelet-poor plasma, concentrated platelet-poor plasma, platelet-rich plasma, and combinations thereof;
obtaining stromal cells compatible with the subject;
combining said stromal cells and said blood component to form a therapeutic composition; and
administering said therapeutic composition to the tissue defect.

2. The method for treating a tissue defect in a human subject according to claim 1, wherein fractionating said blood to produce a blood component comprises centrifugation of said blood to form said blood component.

3. The method for treating a tissue defect in a human subject according to claim 2, wherein centrifugation of said blood to form said blood component comprises centrifuging said blood in a container including at least one buoy that is able to separate said blood into three or more fractions having different densities.

4. The method for treating a tissue defect in a human subject according to claim 3, wherein centrifuging blood in a container comprises displacing a buoy during centrifugation so as to separate said blood into platelet-poor plasma, platelet-rich plasma, and red blood cells.

5. The method for treating a tissue defect in a human subject according to claim 1, wherein said stromal cells are isolated from tissue obtained from said subject.

6. The method for treating a tissue defect in a human subject according to claim 1, wherein said stromal cells are washed at least once with a fluid selected from the group consisting of platelet-rich plasma, platelet-poor plasma, concentrated platelet-poor plasma, whole blood, and combinations thereof.

7. The method for treating a tissue defect in a human subject according to claim 1, wherein the isolated stromal cells are able to differentiate into cells selected from the group consisting of chondrocytes, endothelial cells, osteoblasts, myocytes, neural cells, glial cells, adipocytes, pericytes, cardiomyocytes, epithelial cells, fibroblasts, and combinations thereof.

8. The method for treating a tissue defect in a human subject according to claim 1, further comprising administering to the tissue defect an additive selected from the group consisting of a cytokine, bioactive material, scaffold, buffer, or combinations thereof.

9. The method for treating a tissue defect in a human subject according to claim 1, further comprising administering to the tissue defect a platelet activator selected from the group consisting of thrombin, autologous thrombin, CaCl₂, a coagulation factor, or combinations thereof.

10. The method for treating a tissue defect in a human subject according to claim 1, wherein the defect is a diabetic ulcer, a defect associated with peripheral vascular disease, cardiac muscle damage associated with myocardial infarct, tendinosis, a defect at a site of chemotherapy and/or radiotherapy, or a cartilage defect.

11. A method for treating a tissue defect in a human subject comprising:
obtaining blood and stromal cells compatible with the subject;
combining said blood and said stromal cells;
centrifuging said blood and said stromal cells so that said stromal cells sediment with a blood component comprising platelet-rich plasma;
isolating said stromal cells and said blood component to form a therapeutic composition; and
administering said therapeutic composition to said tissue defect.

12. The method for treating a tissue defect in a human subject according to claim 11, further comprising washing said stromal cells with platelet-poor plasma or concentrated platelet-poor plasma prior to combining said blood and said stromal cells.

13. The method for treating a tissue defect in a human subject according to claim 11, wherein obtaining blood and stromal cells compatible with the subject comprises:
drawing blood from the subject; and
isolating stromal cells from adipose tissue harvested from the subject.

14. A method for treating a tissue defect in a human subject according to claim 13, wherein isolating stromal cells from adipose tissue harvested from the subject comprises:
enzymatically digesting said adipose tissue;
separating stromal cells from adipocytes; and
washing said stromal cells with platelet-poor plasma, concentrated platelet-poor plasma, platelet-rich plasma, or whole blood.

15. The method for treating a tissue defect in a human subject according to claim 11, wherein obtaining blood and stromal cells compatible with the subject comprises:
drawing blood from the subject; and
isolating stromal cells from bone marrow aspirate harvested from the subject.

16. A method for treating a tissue defect in a human subject according to claim 11, wherein administering said therapeutic composition to the tissue defect further comprises administering an additive selected from the group consisting of a cytokine, bioactive material, scaffold, platelet activator, and combinations thereof.

17. The method for treating a tissue defect in a human subject according to claim 11, further comprising administering to the tissue defect a platelet activator selected from the group consisting of thrombin, CaCl₂, a coagulation factor, or combinations thereof.

18. The method for treating a tissue defect in a human subject according to claim 11, further comprising washing said stromal cells with platelet-poor plasma or concentrated platelet-poor plasma prior to combining said blood and said stromal cells.

19. A therapeutic composition for treating a tissue defect, comprising:
isolated stromal cells; and
a blood component, wherein said blood component is selected from the group consisting of platelet-poor plasma, concentrated platelet-poor plasma, platelet-rich plasma, whole blood, and combinations thereof.

20. The therapeutic composition for treating a tissue defect according to claim 19, wherein said isolated stromal cells are cultured cells.

21. The therapeutic composition for treating a tissue defect according to claim 19, wherein said isolated stromal cells and said blood component are derived from the same subject.

22. The therapeutic composition for treating a tissue defect according to claim 19, wherein the stromal cells are selected from the group consisting of adipose stromal cells, bone marrow derived stromal cells, and combinations thereof.

23. The therapeutic composition for treating a tissue defect according to claim 19, wherein said isolated stromal cells are able to differentiate into cells selected from the group consisting of chondrocytes, endothelial cells, osteoblasts, myocytes, neural cells, glial cells, adipocytes, pericytes, cardiomyocytes, epithelial cells, fibroblasts, and combinations thereof.

24. The therapeutic composition for treating a tissue defect according to claim 19, further comprising an additive selected from the group consisting of a cytokine, bioactive material, scaffold, buffer, or combinations thereof.
